(19) United States
(12) Patent Application Publication
(10) Pub. No.: US 2025/0231262 A1
(43) Pub. Date: Jul. 17, 2025

(54) Magnetic Resonance Device for Transportation by Means of Standardized Access Paths

(71) Applicant: Siemens Healthineers AG, Forchheim (DE)

(72) Inventor: Stephan Biber, Erlangen (DE)

(73) Assignee: Siemens Healthineers AG, Forchheim (DE)

(21) Appl. No.: 19/023,524

(22) Filed: Jan. 16, 2025

(30) Foreign Application Priority Data
Jan. 17, 2024 (EP) 24152289.5

(51) Int. Cl. G01R 33/3815; A61B 5/00; A61B 5/055; G01R 33/385

(52) U.S. Cl. CPC G01R 33/3815; A61B 5/055; A61B 5/7475; G01R 33/385; A61B 5/704

(57) ABSTRACT

A magnetic resonance device is provided comprising a retaining structure and a field generation unit having a main magnet, a gradient system and a radiofrequency system, wherein the retaining structure is embodied to mechanically support the main magnet, and wherein the field generation unit is enclosed on its outer circumference by a volume delimited by the retaining structure.

### CROSS-REFERENCE TO RELATED APPLICATIONS

The present application claims priority to and the benefit of European patent application no. EP 24152289.5, filed on Jan. 17, 2024, the contents of which are incorporated herein by reference in their entirety.

### TECHNICAL FIELD

The present disclosure is directed to a magnetic resonance device and, in particular, to a magnetic resonance device comprising a retaining structure and a field generation unit having a main magnet, a gradient system, and a radiofrequency system, with the retaining structure being implemented to mechanically support the main magnet, and the field generation unit being enclosed on its outer circumference by a volume delimited by the retaining structure.

### BACKGROUND

Conventional magnetic resonance devices for medical diagnostic purposes weigh several metric tons and possess dimensions on the order of approximately 2 m in height and 1.6 m in length. On account of these properties, such devices are able to be installed only at sites meeting specific requirements in terms of access and transportation options, as well as load-bearing capacity of floors or ceilings. For example, an examination room in which the magnetic resonance device is set up must possess a sufficiently high ceiling and appropriately arranged access paths. Furthermore, the load-bearing capacity of the ceilings of the examination room must be able to permanently withstand the weight of the magnetic resonance device.

In particular, smaller medical institutions and practices that hold out the prospect of a beneficial application of magnetic resonance imaging methods in new markets (such as e.g. dentistry, neurology, orthopedics) outside of traditional clinical radiology are often not equipped for the installation of conventional magnetic resonance devices. For example, the transportation and installation of a magnetic resonance device during the ongoing operation of a dental practice is difficult, since customers are unwilling to accept any interruption in operation due to building works or conversions, such as e.g. opening up walls or increasing the size of doorways.

For new markets, the costs of a magnetic resonance device, which are affected in particular by the length of a patient receiving zone formed in the main magnet, are also very important. Basically, the main magnet of a magnetic resonance device having a predetermined homogeneity volume becomes more expensive, the shorter it becomes.

Furthermore, conventional magnetic resonance devices comprise parts that project beyond the main magnet and are permanently joined to the latter or cannot easily be disassembled (e.g. parts of the body coil and/or gradient coil, as well as connectors and carrier structures). Such parts increase the dimensions of the magnetic resonance device and can make the transportation and installation of the magnetic resonance device significantly more difficult. Given equivalent requirements in terms of image quality, shortening the main magnet has a negative impact on the cost of the main magnet.

### SUMMARY

It is therefore an object of the disclosure to provide a magnetic resonance device that permits transportation by means of standardized access paths, without reducing image quality and/or increasing costs. This object is achieved according to the embodiments of the disclosure as discussed herein, including the claims.

The magnetic resonance device according to the disclosure comprises a retaining structure and a field generation unit having a main magnet, a gradient system, and a radiofrequency system.

The magnetic resonance device may be embodied to perform a magnetic resonance measurement of an object that is positioned within an image acquisition zone of the magnetic resonance device. The magnetic resonance device may for example be embodied to acquire magnetic resonance data from the object contained within the image acquisition zone. The magnetic resonance device may be further embodied to acquire magnetic resonance image data, such as diagnostic magnetic resonance image data, of the object positioned within the image acquisition zone. The object may be a patient, for example a human being or an animal.

The gradient system may comprise one or more gradient coils. The radiofrequency system may comprise a radiofrequency coil, e.g. a body coil permanently integrated in the magnetic resonance device. In an embodiment, the gradient coil(s) and the radiofrequency coil have electrical conductor structures, which are embodied in a shell or cylinder shape and enclose the image acquisition zone of the magnetic resonance device along a patient access direction. It is conceivable that the one or more gradient coils enclose the radiofrequency coil along the patient access direction.

In an embodiment, the magnetic resonance device according to the disclosure is embodied as a closed-bore scanner or a scanner having a cylindrically shaped patient tunnel. A closed-bore scanner may have a substantially cylindrically shaped image acquisition zone. The main magnet of the closed-bore scanner may comprise one or more magnetic coils which enclose the image acquisition zone along an axial direction or an axis of rotation, e.g. an axis of rotational symmetry, of the main magnet. A magnetic coil may comprise an electrical conductor having negligible electrical resistance at (or under) a superconducting temperature. A direction of a main magnetic field that is provided by means of the main magnet may be aligned substantially parallel to an access direction to the image acquisition zone and/or to the axial direction of the cylindrical bore.

It is equally conceivable that the magnetic resonance device according to the disclosure is embodied as an open-bore scanner. An open-bore scanner may comprise two magnets, which are disposed opposite each other, separated by the image acquisition zone. One direction of the main magnetic field of the open-bore scanner can be oriented substantially orthogonally to an access direction to the image acquisition zone and/or to the axial direction of the cylindrical bore. In an open-bore scanner, a volume delimited by the retaining structure may constitute an imaginary envelope enclosing both magnets as well as the retaining structure, the gradient system, and the radiofrequency system within a smallest possible volume.

The main magnet of the magnetic resonance device may comprise or consist of one or more electromagnets or superconducting magnets. In an embodiment, the main magnet comprises or consists of one or more cylinder-shaped superconducting magnets or superconducting coils. The main magnet may be mechanically coupled to the retaining structure and/or secured in place on the retaining structure. The retaining structure may be embodied to carry and/or support the main magnet. The term “main magnet” may comprise one or more magnets or coils, as well as a dedicated supporting structure for the magnets or coils.

The concepts described herein may also be applied to main magnets that comprise permanent magnets or consist of permanent magnets.

According to the disclosure, the retaining structure is embodied to mechanically support the main magnet. The retaining structure may be further embodied to include the main magnet in a vacuum zone enclosed on its outer circumference by the retaining structure.

The magnetic resonance device according to the disclosure may have an outer vacuum chamber. The outer vacuum chamber may constitute a container which is impervious to fluids, e.g. liquid or gaseous cryogens. The outer vacuum chamber may be embodied to maintain a vacuum in a vacuum zone enclosed by the outer vacuum chamber. In an embodiment, the outer vacuum chamber encloses the main magnet in the vacuum zone. The outer vacuum chamber may of course include further components of the magnetic resonance device, such as e.g. a cryogenic vessel, in the vacuum zone.

The outer vacuum chamber may be embodied as a part of the retaining structure of the main magnet. The main magnet may be mechanically coupled to the outer vacuum chamber or secured to the latter by means of dedicated fastening elements. It is conceivable that the outer vacuum chamber defines or specifies an external shape of the retaining structure and/or of the volume delimited by the retaining structure.

The outer vacuum chamber may comprise an outer wall, an inner wall, and front walls, which mechanically connect the outer wall and the inner wall. The outer wall and the inner wall may be embodied in a shell or cylinder shape. For instance, the outer vacuum chamber may be embodied as a double-walled hollow cylinder which encloses the main magnet in the vacuum zone between the outer wall, the inner wall, and ring-shaped front walls. A cylinder axis of the outer vacuum chamber may be aligned parallel to a cylinder axis of the main magnet or correspond to the cylinder axis of the main magnet. The inner wall of the outer vacuum chamber may correspond to a wall of the patient tunnel of the magnetic resonance device. In an embodiment, the gradient system and/or the radiofrequency system may be secured to and/or supported by the inner wall of the outer vacuum chamber.

In an embodiment, the magnetic resonance device according to the disclosure is embodied as a “dry” system. A “dry” system may contain a small amount of cryogen or no cryogen at all. For example, the magnetic resonance device according to the disclosure may comprise one or more small cryogenic vessels which are thermally connected to the main magnet by means of a heat-conducting structure. A cryogenic vessel of a “dry” system may contain a volume of less than any suitable volume of cryogen, such as for instance less than 10 l, less than 5 l, less than 1 l, etc. of cryogen. In an embodiment, cryogenic vessels are dispensed with. In this case, the main magnet is cooled completely by means of a heat-conducting structure.

In an alternative embodiment, the magnetic resonance device is embodied as a “wet” system. A “wet” system may comprise at least one cryogenic vessel having a volume greater than a suitable established threshold, such as more than 10 l for instance. In “wet” systems, the main magnet may be arranged inside the cryogenic vessel and cooled directly by means of the cryogen.

A cryogen can constitute a fluid having a low boiling point, such as e.g. argon, nitrogen, neon, helium, or the like. A cooling temperature of the cryogen may substantially correspond to a superconducting temperature of the main magnet.

It is conceivable that components of the magnetic resonance device, such as e.g. the main magnet, the cryogenic vessel, the thermal shield, etc., are thermally connected to a cryocooler. In an embodiment, the components of the magnetic resonance device are thermally connected or coupled to the cryocooler by means of a heat-conducting structure (e.g. a thermally conductive solid material or a thermally conductive metal), a convection circuit and/or a heat pipe.

According to the disclosure, the field generation unit is enclosed on its outer circumference by a volume delimited by the retaining structure.

The field generation unit may comprise a plurality of components, such as e.g. the main magnet, the gradient system, and the radiofrequency system. In an embodiment, the field generation unit also comprises connecting elements and/or carrier structures connected to the gradient system and/or the radiofrequency system.

In one embodiment, the field generation unit comprises at least one connecting element and/or a carrier structure which is enclosed on its outer circumference by the volume delimited by the retaining structure.

Within the scope of this disclosure, the cryocooler is deemed to be an autonomous component and consequently may not count as one of the components of the field generation unit.

The field generation unit may be fully encompassed within a volume enclosed by the retaining structure.

A volume delimited or enclosed by the retaining structure may constitute a volume of an imaginary envelope, which encloses the retaining structure on its outer circumference within a smallest possible volume. A shape of the volume delimited or enclosed by the retaining structure may coincide with an external profile of the retaining structure.

In an embodiment, the components of the field generation unit are enclosed on its outer circumference along a patient access direction by the volume delimited by the retaining structure.

The components of the field generation unit may be arranged inside the magnetic resonance device in such a way as to avoid a part of the field generation unit projecting or protruding beyond the retaining structure in the axial direction of the main magnet and/or along a patient access direction.

In an embodiment, the components of the field generation unit may be arranged inside the magnetic resonance device in such way as to avoid a part of the field generation unit projecting or protruding beyond an end or a side of the retaining structure.

In an embodiment, the magnetic resonance device is embodied as a closed-bore scanner. A length of the retaining structure along the cylinder axis of the main magnet may be any suitable length, such as for instance less than 80 cm, less than 79 cm, less than 78 cm, less than 77 cm, etc., wherein a length of the field generation unit corresponds to or is less than the length of the retaining structure along the cylinder axis of the main magnet. The retaining structure may e.g. define a length of the main magnet that is available for the main magnet along the cylinder axis of the main magnet.

By avoiding parts of the field generation unit that project beyond a dimension or an end of the retaining structure, it is advantageously possible to optimize a size of the main magnet and consequently a quality of the homogeneous volume of the magnetic resonance device according to the disclosure for access paths having standardized dimensions, such as e.g. corridors and doors. As a result, the magnetic resonance device can also be installed in smaller medical institutions and practices and provide high-quality image data.

In an embodiment, a shortening of the main magnet to allow for protruding connecting elements and/or carrier structures of the field generation unit can be avoided by means of a magnetic resonance device according to the disclosure. As a result, a length of the main magnet can advantageously be increased or optimized along the patient access direction. Furthermore, additional costs associated with a shortening of the main magnet while maintaining the same quality of the imaging volume can advantageously be avoided or reduced by means of the magnetic resonance device according to the disclosure. The magnetic resonance device according to the disclosure can additionally enable a relationship between dimensions and costs of the main magnet to be improved or optimized.

In one embodiment of the magnetic resonance device according to the disclosure, the radiofrequency system comprises a carrier structure, which is embodied to couple a radiofrequency coil of the radiofrequency system mechanically to the retaining structure.

The carrier structure may be embodied to carry the radiofrequency coil of the radiofrequency system and/or to provide a mechanical support for the radiofrequency coil. It is conceivable that the carrier structure is embodied to secure the radiofrequency coil in position on the retaining structure.

The carrier structure may comprise any suitable mechanical element, such as e.g. a strut, a rod, a bar, a bracket, an L element, a T element, a V element, or the like. The carrier structure can be secured to the radiofrequency coil and/or the retaining structure by means of any suitable mechanical connection. A suitable mechanical connection may comprise a positive-locking connection, a force-fitting connection, and/or a material-to-material bonded connection. For example, a mechanical connection between the carrier structure and the radiofrequency coil, as well as a mechanical connection between the carrier structure and the retaining structure, may comprise a screwed connection, a bolted connection, a clamped connection and/or an adhesive bond.

In an embodiment, the carrier structure of the radiofrequency system is housed in a recess in a gradient coil of the gradient system or is led through (e.g. routed through) the recess in a gradient coil of the gradient system. It is conceivable that the radiofrequency coil is separated or spaced apart from the retaining structure of the main magnet by means of a gradient coil of the gradient system. A recess in the gradient system may constitute e.g. a cutout or a hole in a gradient coil. The carrier structure may e.g. project through the recess in the gradient system and mechanically connect the radiofrequency coil to the retaining structure of the main magnet. It is conceivable that the recess in the gradient system is arranged in a plane or a curved surface of an electrical conductor structure of a gradient coil of the gradient system. The electrical conductor structure of the gradient coil may be arranged around the recess or border the recess.

The carrier structure is enclosed on its outer circumference by the volume delimited by the retaining structure.

The carrier structure may be completely surrounded by the volume delimited by the retaining structure so as to prevent the carrier structure protruding beyond an end or a side of the retaining structure.

By providing a magnetic resonance device according to the disclosure it is advantageously possible to prevent a carrier structure of the radiofrequency system projecting or protruding beyond an end of the retaining structure of the main magnet. As a result, a dimension of the main magnet along a patient access direction can advantageously be increased or optimized.

In one embodiment of the magnetic resonance device according to the disclosure, the retaining structure comprises an outer vacuum chamber, which encloses the main magnet on its outer circumference. A wall of the outer vacuum chamber has an indentation, a section of the carrier structure of the radiofrequency system being at least partly housed in the indentation of the outer vacuum chamber.

The outer vacuum chamber may be embodied according to an above-described embodiment.

The indentation in the wall of the outer vacuum chamber may constitute a depression, a trough and/or a cutout in the material of the wall of the outer vacuum chamber. In an embodiment, the wall of the outer vacuum chamber is not breached by the indentation.

The indentation in the wall of the outer vacuum chamber can provide a volume which is used for the mechanical connection to the carrier structure of the radiofrequency system, but also to a carrier structure of the gradient system. In one example, the carrier structure of the radiofrequency system, as well as a carrier structure of the gradient system, may be mechanically secured or anchored in place in the indentation in the wall of the outer vacuum chamber.

In one embodiment of the magnetic resonance device, the gradient system, and the radiofrequency system are mechanically connected to the retaining structure and/or secured in position on the retaining structure by means of a carrier structure.

By providing an indentation in a wall of the outer vacuum chamber, it is possible for the carrier structure of the radiofrequency system to be mechanically connected to the retaining structure of the main magnet. As a result, a required dimension of the recess in the gradient system can advantageously be reduced or minimized.

In a further embodiment of the magnetic resonance device, the radiofrequency system has a provisional carrier structure. The provisional carrier structure is embodied to secure a radiofrequency coil of the radiofrequency system in place in a reversible manner to a gradient coil of the gradient system and/or to the retaining structure.

The provisional carrier structure can be connected to the gradient system and/or the retaining structure by means of any suitable force-fitting and/or positive-locking mechanical connection. For example, the provisional carrier structure may be connected to the gradient coil and/or the retaining structure by means of a screwed connection, a clamped connection and/or a bolted connection. The provisional carrier structure can be mechanically connected in a comparable manner to the radiofrequency coil. The provisional carrier structure may be embodied to secure the radiofrequency coil of the radiofrequency system, and optionally also the gradient coil of the gradient system, in the interim or temporarily to the retaining structure. The provisional carrier structure may comprise a mechanical element according to the above-described carrier structure. The provisional carrier structure may be embodied as a transit bolt or transit screw.

In one embodiment, the provisional carrier structure mechanically connects the radiofrequency coil of the radiofrequency system to the gradient coil of the gradient system, the gradient system being mechanically connected to the retaining structure of the main magnet.

In a further embodiment, the provisional carrier structure projects through a recess in the gradient coil through the gradient system and connects the radiofrequency coil of the radiofrequency system, and optionally the gradient coil of the gradient system, to the retaining structure of the main magnet.

According to the disclosure, the provisional carrier structure is embodied as reversibly removable and, in a purpose-built arrangement for securing the radiofrequency coil of the radiofrequency system to the gradient coil of the gradient system and/or to the retaining structure, is enclosed on its outer circumference by the volume delimited by the retaining structure.

It is conceivable that following the transportation of the magnetic resonance device, the provisional carrier structure is replaced by a conventional carrier structure or a carrier structure according to an above-described embodiment.

A provisional carrier structure may be designed to withstand mechanical forces occurring during the transportation of the magnetic resonance device. This advantageously enables a necessary mechanical anchoring in the retaining structure of the main magnet and/or a recess in the gradient system to be dimensioned smaller. Following the transportation of the magnetic resonance device, the provisional carrier structure can be replaced by a conventional carrier structure, which is also able to withstand (electromagnetic) forces that occur during the operation of the magnetic resonance device.

In an embodiment of the magnetic resonance device, a radiofrequency coil of the radiofrequency system comprises a connecting element, which is embodied to connect the radiofrequency coil to a power source and/or to an external cooling system.

The connecting element of the radiofrequency coil of the radiofrequency system may comprise an electrical connector. The electrical connector may be implemented for example as a pin, a terminal, a plug, or a socket. The electrical connector may be embodied to connect an electrical conductor structure of the radiofrequency coil to a power source, e.g. to a radiofrequency unit of the magnetic resonance device.

The connecting element of the radiofrequency coil may further comprise a cooling connection. A cooling connection may comprise e.g. a pipe union, a hose connection, or a connection nozzle. The cooling connection may be embodied to supply a cooling circuit of the radiofrequency system with a coolant and/or to thermally couple the cooling circuit of the radiofrequency system to an external cooling circuit.

According to the disclosure, the connecting element of the radiofrequency coil is enclosed on its outer circumference by the volume delimited by the retaining structure.

In one embodiment, the connecting element of the radiofrequency coil is arranged in the indentation in the wall of the outer vacuum chamber according to an above-described embodiment. However, it is also conceivable that the connecting element of the radiofrequency coil is arranged in a recess in a gradient coil of the gradient system. Furthermore, the connecting element of the radiofrequency coil may also extend into a patient receiving zone of the magnetic resonance device.

In conventional magnetic resonance devices, electrical connectors and/or cooling connections typically project beyond an end or a footprint of the outer vacuum chamber, for which reason the main magnet must be appropriately shortened for transportation by means of standardized access paths. By providing a radiofrequency coil with a connecting element according to the disclosure it is advantageously possible to increase or optimize a dimension of the main magnet.

In a further embodiment of the magnetic resonance device according to the disclosure, the field generation unit comprises a connecting element, which is embodied to connect a radiofrequency coil of the radiofrequency system and/or a gradient coil of the gradient system to an external power source and/or an external cooling system. The connecting element of the field generation unit is implemented as a flexible connecting element, and is embodied to be positioned relative to the main magnet and to be stowed within the volume delimited by the retaining structure.

The flexible connecting element may be embodied as movable. For example, the flexible connecting element may comprise a movable hose and/or a movable electric cable. The flexible connecting element may further comprise an electrical connector and/or a cooling connection. The electrical connector and/or the cooling connection may be arranged at a connecting end of the flexible connecting element.

In an embodiment, the flexible connecting element is embodied as movable in such a way that the connecting end of the flexible connecting element can be brought out if need be from the volume enclosed by the main magnet with the retaining structure. Similarly, the flexible connecting element may be embodied to be stowed in the volume delimited by the retaining structure.

By providing a flexible connecting element, which can be stowed temporarily in the volume delimited by the retaining structure, it is advantageously possible to prevent components of the field generation unit protruding beyond an end of the retaining structure of the main magnet during the transportation of the magnetic resonance device.

In one embodiment of the magnetic resonance device according to the disclosure, the connecting element projects into a volume enclosed by the radiofrequency coil and/or into a patient receiving zone of the magnetic resonance device.

The magnetic resonance device may be embodied as a closed-bore scanner comprising a cylinder-shaped main magnet. The cylinder-shaped main magnet may enclose the image acquisition zone and/or the patient receiving zone circumferentially along the patient access direction and/or along a main magnetic field direction. The connecting element of the radiofrequency coil may for example be oriented in a radial direction of the main magnet. For instance, the connecting element of the radiofrequency coil may project into the patient receiving zone in the radial direction of the main magnet.

In an open-bore scanner comprising two separate magnets, the connecting element of the radiofrequency coil may project into the patient receiving zone that extends between the two magnets.

The connecting element of the radiofrequency coil may be arranged in the patient receiving zone in such a way that a blocking of the patient access to the magnetic resonance device is avoided. For example, the connecting element of the radiofrequency coil may be arranged at a second end of the magnetic resonance device which is disposed opposite a first end of the magnetic resonance device having the patient access.

An above-described arrangement of the connecting element of the radiofrequency coil advantageously enables a dimension of the main magnet to be increased or maximized compared to conventional magnetic resonance devices.

In an embodiment of the magnetic resonance device according to the disclosure, a gradient coil of the gradient system comprises a connecting element. The connecting element of the gradient coil is embodied to connect the gradient system to an external power source and/or an external cooling system. The connecting element of the gradient coil is enclosed on its outer circumference by the volume delimited by the retaining structure.

The connecting element of the gradient coil may be embodied according to an embodiment of the connecting element of the radiofrequency coil. For instance, the connecting element of the gradient coil may comprise an electrical connector and/or a cooling connection.

It is conceivable that the gradient coil of the gradient system has a recess, which is embodied to house the connecting element of the gradient coil.

It is furthermore conceivable that the connecting element of the gradient coil is arranged and/or anchored at least partially in an indentation in the wall of the outer vacuum chamber.

In an embodiment of the magnetic resonance device according to the disclosure, a radiofrequency coil of the radiofrequency system and/or the gradient coil of the gradient system have/has a recess which is embodied to house the connecting element of the gradient coil.

In an embodiment, the radiofrequency coil of the radiofrequency system has a recess which is embodied to house at least a part of the connecting element of the gradient coil.

In an embodiment, the connecting element of the gradient coil is mechanically integrated with the connecting element of the radiofrequency coil.

It is conceivable that the gradient coil of the gradient system and/or the radiofrequency coil of the radiofrequency system have/has recesses which are embodied to house the connecting element of the gradient coil, the connecting element of the radiofrequency coil and/or a combined connecting element comprising the connecting element of the gradient coil and the connecting element of the radiofrequency coil.

In an embodiment, the connecting element of the radiofrequency coil and/or the connecting element of the gradient coil are/is mechanically secured or anchored in place in the recess of the gradient coil and/or the recess of the radiofrequency coil.

In a further embodiment of the magnetic resonance device according to the disclosure, the connecting element of the gradient coil is led through a recess in the radiofrequency coil and projects into a volume enclosed by the radiofrequency coil and/or into a patient receiving zone of the magnetic resonance device.

Analogously to the connecting element of the radiofrequency coil, the connecting element of the gradient coil may project into the volume enclosed by the radiofrequency coil and/or into the patient receiving zone of the magnetic resonance device. In this case the connecting element of the gradient coil may be led through a plane or shell of the radiofrequency coil comprising the conductor structure.

The connecting element of the gradient coil shares the advantages of the connecting element of the radiofrequency coil.

In a further embodiment, the magnetic resonance system according to the disclosure comprises a reversibly removable gradient connecting plate, which is embodied to connect a gradient coil of the gradient system and/or a radiofrequency coil of the radiofrequency system electrically and mechanically to a power source.

The reversibly removable gradient connecting plate may be embodied to mechanically secure an electrical connecting lead, which electrically connects the gradient coil to the power source. The removable gradient connecting plate may e.g. be embodied to stabilize the electrical connecting lead against Lorentz forces occurring during operation of the magnetic resonance device. It is conceivable that the removable gradient connecting plate comprises a rigid material, which is embodied to prevent a deformation of the removable gradient connecting plate and/or the electrical connecting lead due to electromagnetic forces.

The power source may constitute an external power source. However, the power source may constitute a gradient control unit and/or a radiofrequency unit of the magnetic resonance device.

The reversibly removable gradient connecting plate may be arranged on the magnetic resonance device outside of the volume delimited by the retaining structure. For instance, the removable gradient connecting plate may be reversibly secured or mounted on the retaining structure of the main magnet. The reversibly removable gradient connecting plate may e.g. be secured on the retaining structure of the main magnet by means of a reversible mechanical connection, such as via a force-fitting and/or positive-locking connection. For example, the removable gradient connecting plate may be secured on the retaining structure of the main magnet by means of a screwed connection, a bolted connection or a clamped connection.

By providing a reversibly removable gradient connecting plate, it is possible to reduce or minimize an external dimension of the magnetic resonance device temporarily, e.g. for the transportation and/or the installation of the magnetic resonance device. As a result, a dimension of the main magnet can advantageously be increased or maximized.

In one embodiment, the magnetic resonance device according to the disclosure comprises a retainer, which is embodied to hold the magnetic resonance device at a predetermined distance from a floor surface.

The retainer may comprise any mechanical structure, which is embodied to secure the magnetic resonance device in a predetermined position relative to the floor surface. The retainer may for instance be embodied to hold the main magnet in a predetermined orientation to the floor surface. In an embodiment, the retainer carries the main magnet. The retainer may be mechanically connected to the retaining structure of the main magnet.

The retainer can be connected to any wall, e.g. to the floor surface, to a ceiling, and/or to a side wall, of an examination room by means of a suitable mechanical connection.

According to the disclosure, a part of the retainer that exceeds a dimension of the retaining structure in one spatial direction is embodied as reversibly removable.

The reversibly removable part of the retainer may be connected to the retainer by means of any desired suitable mechanical connection. For example, the reversibly removable part of the retainer may be connected to the retainer by means of a force-fitting and/or a positive-locking mechanical connection, such as e.g. a screwed connection, a clamped connection, and/or a bolted connection.

It is conceivable that the reversibly removable part of the retainer, when connected in accordance with its intended use to the retainer in at least one spatial direction, e.g. in at least two spatial directions, projects beyond the volume delimited by the retaining structure. For example, the retainer may project in height and in length or in width beyond the volume delimited by the retaining structure. The reversibly removable part of the retainer may be embodied in such a way that once the reversibly removable part of the retainer has been removed, the retainer projects or protrudes in one spatial direction only, e.g. in height, beyond the volume enclosed or delimited by the retaining structure.

The reversibly removable part of the retainer may comprise a section of the retainer or the entire retainer. For example, the entire retainer may be embodied to be reversibly fastened to the retaining structure.

In a further embodiment, the magnetic resonance device according to the disclosure comprises a retainer, which is embodied to hold the magnetic resonance device at a predetermined distance from the floor surface, the retainer being embodied as rotatable and/or pivotable relative to the main magnet.

The retainer may be embodied in accordance with an above-described embodiment.

It is conceivable that the retainer projects or protrudes beyond the volume delimited by the retaining structure in a spatial direction which is limiting during the transportation of the magnetic resonance device. For example, the retainer may project widthwise beyond the volume enclosed by the retaining structure, the width of the magnetic resonance device being limited by a width of a standardized access path (e.g. a door or corridor).

The retainer may be rotatably connected to the retaining structure to allow a temporary reduction in the width of the magnetic resonance device, and consequently enable a transportation by means of the standardized access path.

It is conceivable that the retainer and/or the retaining structure comprise/comprises an articulated joint or a mechanism, which are/is embodied to rotate and/or pivot the retainer relative to the main magnet with the retaining structure. The articulated joint or the mechanism may be embodied e.g. as a sliding contact bearing and/or as a rolling contact bearing. It is conceivable that the articulated joint or the mechanism comprises a radial bearing, a linear bearing, a radial bearing and/or an axial bearing. For example, the articulated joint or the mechanism may comprise a hinge and/or a ball bearing.

A retainer according to the disclosure enables a dimension of the magnetic resonance device to be temporarily reduced, e.g. for the transportation and/or the installation of the magnetic resonance device. As a result, a dimension of the main magnet can advantageously be increased or maximized since the retainer can scale with the dimension of the main magnet, yet can be temporarily reduced in size in at least one spatial direction for transportation purposes.

In an embodiment, the magnetic resonance device according to the disclosure comprises an outer casing having a reversibly removable section.

The outer casing of the magnetic resonance device may constitute any desired housing or cover that envelops technical components of the magnetic resonance device and/or protects them against a mechanical impact from outside. The outer casing may equally be embodied to protect a person from a direct contact with the technical components of the magnetic resonance device.

The outer casing may comprise at least one reversibly removable section. It is conceivable that the reversibly removable section is embodied to be reversibly mechanically connected to the outer casing and/or the retaining structure of the main magnet.

According to the disclosure, a user interface, which is carried by the removable section, is connected to a control unit of the magnetic resonance device by means of an electrical interface, an electrical connecting lead which connects the user interface to the electrical interface being embodied so as to allow a reversible removal of the removable section having the user interface from the magnetic resonance device.

The electrical interface may be embodied to prevent a direct mechanical connection between the user interface and a control unit of the magnetic resonance device by means of electric cables. A length of an electric cable connecting the user interface to the electrical interface may be sufficient to allow the reversibly removable section to be removed from the magnetic resonance device without damaging the electric cable, the user interface, and/or the electrical interface.

The user interface may be referred to herein s user interface circuitry, and may constitute an operator control panel or an HMI (Human-Machine Interface) panel of the magnetic resonance device. It is conceivable that the user interface is mechanically connected to the reversibly removable section of the outer casing.

By providing a reversibly removable section of the outer casing, it is possible to reduce or minimize an external dimension of the magnetic resonance device temporarily, e.g. for the transportation and/or the installation of the magnetic resonance device. As a result, a dimension of the main magnet can advantageously be increased or maximized.

In a further embodiment, the magnetic resonance device according to the disclosure comprises an outer casing, a section of the outer casing enclosing the main magnet along a section of a patient access direction, and a dimension of the section of the outer casing along the patient access direction is less than a dimension of the retaining structure along the patient access direction.

The section of the outer casing may enclose the main magnet, all magnets of the main magnet, the field generation unit, and/or the retaining structure of the magnetic resonance device along the section of the patient access direction.

The section of the outer casing may be embodied in a single piece or be composed of a plurality of parts.

It is conceivable that the magnetic resonance device is embodied as a closed-bore scanner. A length of the section of the outer casing may be less than a length of the retaining structure in the axial direction, e.g. along a cylinder axis, of the magnetic resonance device.

The outer casing may have at least one end section that is embodied to be reversibly mechanically connected to a distal section or an axial end of the retaining structure. The distal section or the axial end of the retaining structure may constitute a base area or a top surface of a cylinder-shaped body of the retaining structure. It is conceivable that the at least one end section of the outer casing encompasses the distal section or the axial end of the retaining structure in such a way that the at least one end section also encloses a section of the main magnet along the patient access direction or the cylinder axis of the main magnet.

In an embodiment, the section of the outer casing terminates with the at least one end section such that the main magnet with the retaining structure is completely enclosed by the outer casing.

The outer casing according to the disclosure allows a reversible removal of sections of the outer casing, e.g. of at least one end section of the outer casing, which in a mounted state on the magnetic resonance device would exceed a measure of a standardized access path. By providing a section of the outer casing which is less than a dimension of the main magnet along a main extension direction it is advantageously possible to avoid a protrusion of the outer casing beyond a cross-sectional area of the magnetic resonance device which is coordinated with a dimension of the standardized access path.

### BRIEF DESCRIPTION OF THE DRAWINGS

Further advantages and details will become apparent from the following description of exemplary embodiments taken in conjunction with the schematic drawings, in which:

FIG. 1 illustrates a conventional magnetic resonance device; and

FIGS. 2-11 illustrate various embodiments and views of an example magnetic resonance device according to the disclosure.

### DETAILED DESCRIPTION OF THE DISCLOSURE

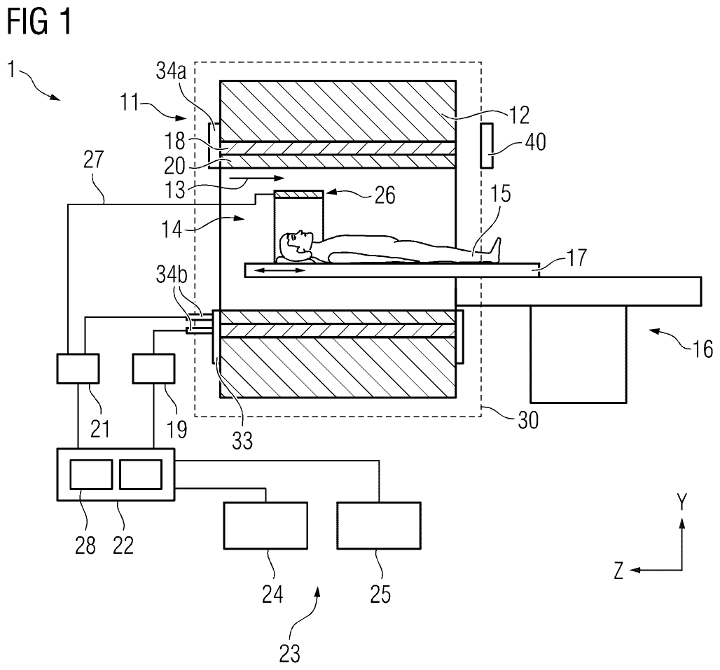

FIG. 1 illustrates a conventional magnetic resonance device 1. The magnetic resonance device 1 comprises a field generation unit 11 (also referred to herein as a field generator), which has a main magnet 12 comprising one or more permanent magnets, electromagnets, or superconducting magnets for generating a strong and homogeneous main magnetic field 13 (B0 magnetic field). In addition, the magnetic resonance device 1 comprises a patient receiving zone 14 for accommodating a patient 15. In the exemplary embodiment shown, the patient receiving zone 14 is embodied in a cylinder shape and is enclosed in a circumferential direction by the main magnet 12. In principle, however, embodiments of the patient receiving zone 14 differing from this example are also conceivable. The patient receiving zone 14 may substantially conform to an image acquisition zone of the magnetic resonance device 1.

In the example shown in FIG. 1, the examination object is a patient 15. The patient 15 can be positioned in the patient receiving zone 14 by means of a patient support and positioning device 16 of the magnetic resonance device 1. For this purpose, the patient support and positioning device 16 comprises a patient table 17 embodied as movable within the patient receiving zone 14.

The field generation unit 11 additionally comprises a gradient system having at least one gradient coil 18 for generating magnetic gradient fields which is used for spatial encoding during a magnetic resonance measurement. The gradient coil 18 is driven by means of a gradient control unit 19 of the magnetic resonance device 1. It is conceivable that the gradient system comprises a plurality of gradient coils 18 for generating magnetic gradient fields in different spatial directions that may be oriented orthogonally to one another.

The field generation unit 11 further comprises a radiofrequency system having a radiofrequency coil which in the present exemplary embodiment is embodied as a body coil 20 permanently integrated in the magnetic resonance device 1. The body coil 20 is configured to excite nuclear spins that are contained in the main magnetic field 13 generated by the main magnet 12. The body coil 20 is driven by a radiofrequency unit 21 of the magnetic resonance device 1 and beams (e.g. transmits or radiates in) radiofrequency excitation pulses into the image acquisition zone, which is substantially formed by the patient receiving zone 14 of the magnetic resonance device 1. The body coil 20 is further embodied to receive magnetic resonance signals and can constitute a receive unit or a part of a receive unit of the magnetic resonance device 1.

The magnetic resonance device 1 comprises a control unit 22 for controlling the magnetic resonance device 1, e.g. the gradient control unit 19 and the radiofrequency unit 21. The control unit 22 is embodied to control an execution of an imaging sequence, such as e.g. a GRE (gradient echo) sequence, a TSE (turbo spin echo) sequence or a UTE (ultra-short echo time) sequence. In addition, the control unit 22 comprises a computing unit 28 for evaluating magnetic resonance signals that are acquired by means of an imaging sequence during a magnetic resonance measurement.

The magnetic resonance device 1 may comprise a user interface 23, which has a signal connection to the control unit 22. Control information, such as e.g. imaging parameters of the magnetic resonance measurement, can be displayed on a display unit 24, for example on at least one monitor, of the user interface 23. The display unit 24 may be configured to provide a graphical user interface with the view of a relevant body region of the patient 15. The user interface 23 additionally has an input unit 25 by means of which parameters of a magnetic resonance measurement can be entered or modified by a user.

The magnetic resonance device 1 may comprise further components, such as e.g. a local coil 26. The local coil 26 may be arranged in a position in accordance with its intended use on a diagnostically or therapeutically relevant body region of the patient 15. The local coil 26 may comprise a plurality of antenna elements, which are embodied to acquire magnetic resonance signals of the relevant body region of the patient 15 and to transmit them to the computing unit 28 and/or the control unit 22. For this purpose, the local coil can be connected to the radiofrequency unit 21 and the control unit 22 by means of an electric connecting lead 27 or another suitable signal connection. Analogously to the body coil 20, the local coil 26 may also be embodied to excite nuclear spins in the jaw region of the patient 15. For this purpose, the local coil 26 can be driven by the radiofrequency unit 21.

Conventional magnetic resonance devices 1 typically comprise components which project or protrude beyond a volume delimited by the retaining structure 32 (see FIG. 9). Such parts may be sections or components of the field generation unit 11, such as e.g. electrical connectors 34b of the gradient coil 18 and/or the body coil 20, carrier structures 33 for the body coil 20, cooling connections 34a for the gradient coil 18 and/or the body coil 20, parts of the outer casing 30, and/or parts of a user interface, such as e.g. an HMI panel on the outer casing 30.

Conventional whole-body magnetic resonance devices 1 are typically transported by means of dedicated lifting devices over predetermined transportation routes to clinical institutions, for which reason an increased external dimension due to projecting parts is not a problem in most cases.

However, in the case of dedicated scanners which, owing to smaller external dimensions, are also to be transported to smaller clinical institutions and practices via standardized access paths, such projecting parts can represent a major problem.

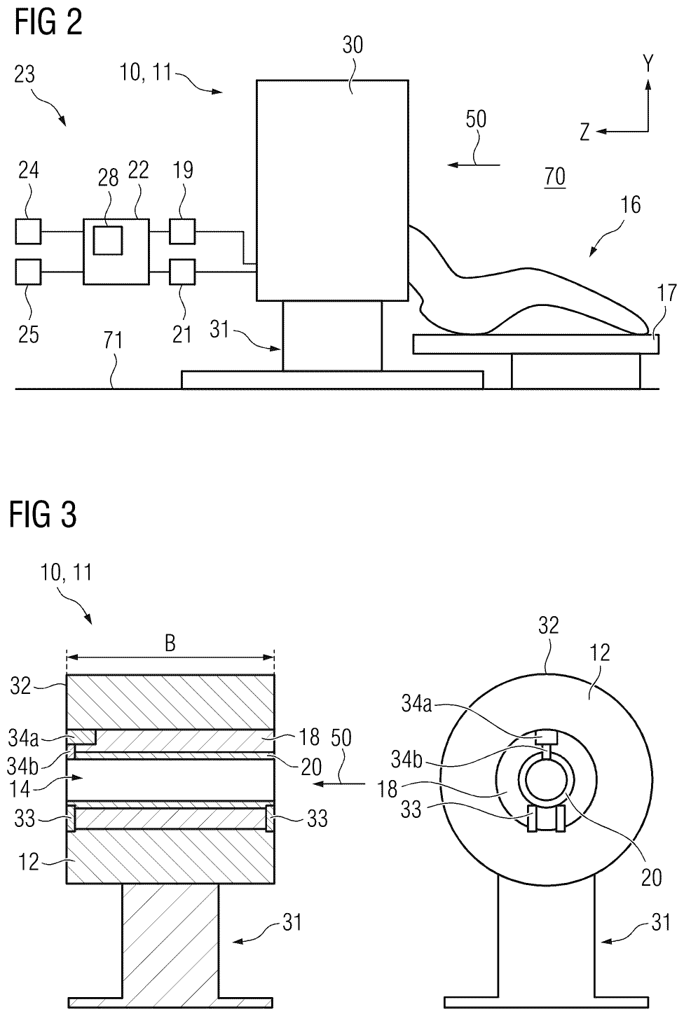

FIG. 2 illustrates an embodiment of a magnetic resonance device 10 according to the disclosure. Basically, the functions and components of the magnetic resonance device 10 shown by way of example in FIG. 2 are consistent with the above-described functions and components of a conventional magnetic resonance device 1 (see FIG. 1).

For example, the magnetic resonance device 10 may be embodied to perform a magnetic resonance examination of a jaw region and/or an eye region of a patient 15. The magnetic resonance device 10 according to the disclosure may also be embodied to conduct a cardiac imaging examination, a mammographic imaging examination, a neurological imaging examination, a urological imaging examination, an orthopedic imaging examination, a prostate imaging examination, or an imaging examination of other body regions of the patient 15.

In the example shown in FIG. 2, the magnetic resonance device 10 is carried by a retainer 31 and held at a predetermined distance from a floor surface 71 of an examination room 70. It is conceivable that the retainer 31 comprises a positioning unit (not shown) which is embodied to position and/or orientate the field generation unit 11 of the magnetic resonance device 10 relative to a diagnostically-relevant body region of the patient 15. For example, the positioning unit may comprise a revolute joint which is embodied to rotate the field generation unit 11 along one direction of rotation. A spatial position of the field generation unit 11 along a Y-direction and/or a Z-direction can be set by way of a suitable telescopic system and/or rail system, which is mechanically coupled to the retainer 31.

It is equally conceivable that the magnetic resonance device 10 comprises a patient support and positioning device 16, as shown in FIG. 2, and/or a patient table 17, which is embodied to position a diagnostically-relevant body region of the patient 15 in the image acquisition zone.

In contrast to the embodiment shown in FIG. 2, the retainer 31 may also be embodied to mount the magnetic resonance device 10 or the field generation unit 11 on a wall and/or a ceiling of an examination room 70.

The magnetic resonance device 10 shown may of course contain further components which magnetic resonance devices typically comprise. The general mode of operation of a magnetic resonance device is well known to the person skilled in the art. A detailed description of further components or of a measurement data acquisition of a magnetic resonance examination is therefore dispensed with.

Instead of the cylinder-shaped design, it is also conceivable that the magnetic resonance device 10 has a field generation unit 11 featuring a C-shaped, triangular, or asymmetric structure. As an example, the magnetic resonance device 10 may be a dedicated scanner, which is embodied to perform a magnetic resonance imaging examination of a jaw region and/or head region of a standing or sitting patient 15. The following FIGS. 3 to 7 show further aspects of the magnetic resonance device 10 according to the disclosure in detail.

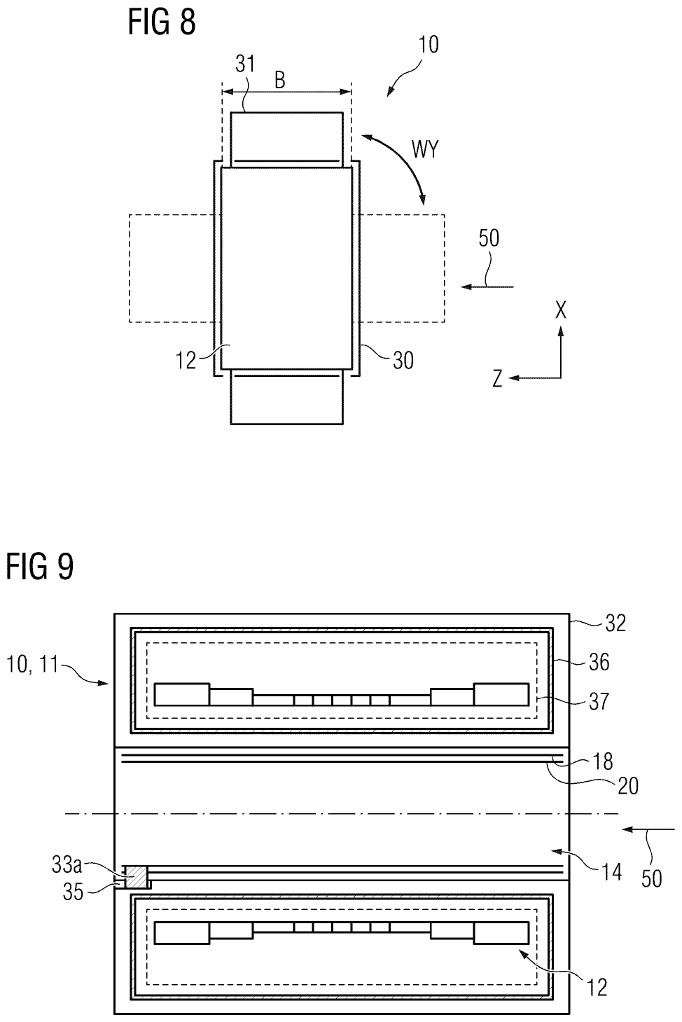

FIG. 3 illustrates an embodiment of the magnetic resonance device 10 according to the disclosure in a cross-sectional view. In the present example, the body coil 20 has a plurality of carrier structures 33, which project through recesses in the gradient coil 18, and mechanically connect the body coil 20 to the retaining structure 32 of the main magnet 12 (cf. FIGS. 8 to 10). This prevents the carrier structures 33 protruding or projecting beyond an axial end of the main magnet 12 and the retaining structure 32.

FIGS. 8 to 10 schematically illustrate a number of possible ways in which the body coil 20 can be secured to the gradient coil 18 and/or the retaining structure 32 by means of a carrier structure 33.

In the example shown in FIG. 3, the cooling connection 34a for the gradient coil 18 and, optionally for the body coil 20, is housed in a recess of the gradient coil 18. Conductor structures of the gradient coil 18 may be placed around the recess in a material of the gradient coil 18 (not shown).

Similarly, an electrical connector 34b, which electrically connects the gradient coil 18 to the gradient control unit 19 (see FIG. 2), is housed in a corresponding recess in the gradient coil 18 and the body coil 20.

The electrical connector 34b, along with the cooling connection 34a, can constitute connecting elements 34, which are housed or accommodated in a corresponding recess in the gradient coil 18 and/or the body coil 20. The connecting elements 34 may be embodied to mechanically secure connected electrical leads as well as cooling connections and/or to stabilize the same against Lorentz forces.

By housing the electrical connectors 34b, the carrier structures 33, and the cooling connections 34a in recesses of the gradient coil 18 and/or the body coil 20, it is possible to reduce a width B of the magnetic resonance device 10 according to the disclosure to a standardized measure, e.g. a width of less than 80 cm. At the same time, the main magnet 12 can be dimensioned in relation to the available width B and consequently provide a higher magnetic field strength and/or an improved homogeneity of the main magnetic field.

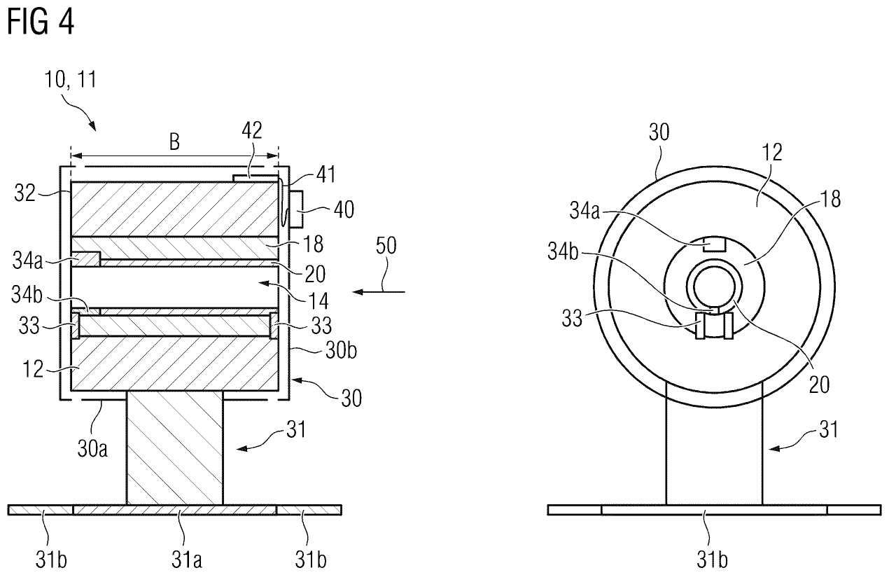

In the embodiment shown in FIG. 4, the electrical connectors 34b of the gradient coil 18 and the body coil 20 are housed in a recess in the body coil 20. However, it is also conceivable for the electrical connectors 34b for the gradient coil 18 and the body coil 20 to be housed or arranged in a recess in the gradient coil 18 and/or in a recess of the body coil 20.

In the present example, the cooling connection 34a of the gradient system is arranged in a recess in the body coil 20. The cooling connection 34a may also be arranged in a recess of the gradient coil 18 and/or the body coil 20.

The embodiment of the magnetic resonance device 10 according to the disclosure shown in FIG. 4 has an outer casing 30 with a reversibly removable section 30b. The reversibly removable section 30b carries a user interface 40 (e.g. user interface circuitry) which is embodied, for example, as an HMI panel or a tablet having a docking station. In an embodiment, the user interface 40 is electrically connected to the control unit 22 of the magnetic resonance device 10 and allows a user to control functions of the magnetic resonance device 10.

The user interface 40 is connected by means of an electrical connecting lead 41 to an electrical interface 42, which is in turn connected to the control unit 22 of the magnetic resonance device 10. The electrical connecting lead 41 is embodied so as to allow the removable section 30b of the outer casing 30 with the user interface 40 to be removed from the magnetic resonance device 10. In an embodiment, the electrical connecting lead 41 has a length that enables the removable section 30b to be removed without mechanically separating the electrical connecting lead 41 from the electrical interface 42 and the user interface 40. The electrical interface 42 may be arranged in such a way that prevents it from projecting beyond a width B of the main magnet and the retaining structure 32 of the main magnet 12. For this purpose, the electrical interface 42 can be arranged on an outside face, e.g. a radial outside face of the retaining structure 32.

Furthermore, the outer casing 30 comprises a section 30a, which encloses the main magnet 12 along a section of the patient access direction 50. In the example shown in FIG. 4, a dimension of the section 30a of the outer casing along the patient access direction 50 is less than a dimension of the main magnet 12 and the retaining structure 32 along the patient access direction 50. It is conceivable that the section 30a can also be secured to the magnetic resonance device 10 during the transportation, since the width B of the magnetic resonance device 10 is not increased by the section 30a of the outer casing 30.

In the example shown in FIG. 4, the magnetic resonance device 10 additionally comprises a retainer 31 having removable parts 31b. The removable parts 31b exceed the width B of the retaining structure 32 along the patient access direction 50 when they are mounted on the retainer 31 in accordance with their intended use. The removable parts 31b of the retainer 31 may be embodied as being reversibly removable. The removable parts 31b can be reversibly connected to the retainer 31 by means of any suitable mechanical connection. By removing the parts 31b from the retainer 31, it is possible to avoid the width B of the magnetic resonance device 10 being exceeded during transportation.

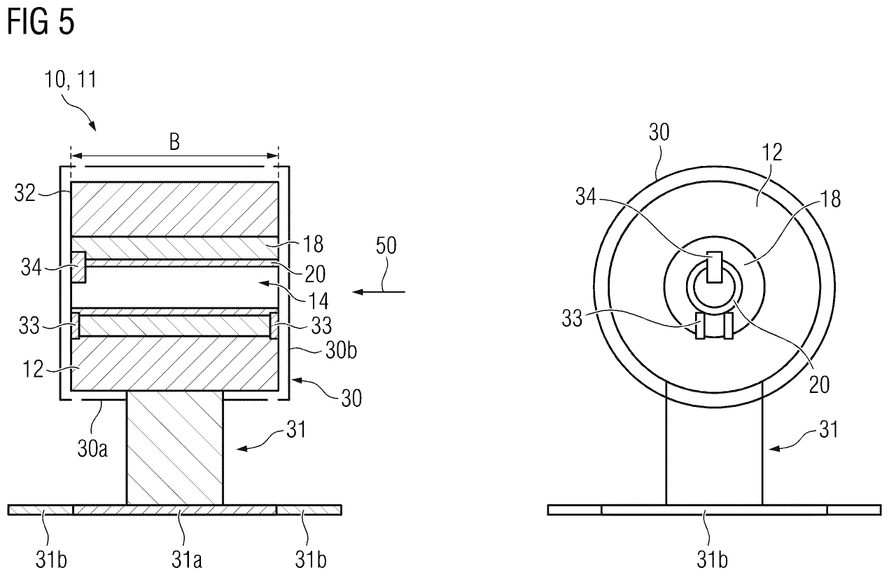

FIG. 5 illustrates a further embodiment of the magnetic resonance device 10 according to the disclosure. In the example shown, one or more connecting elements 34 of the gradient coil 18 and/or the body coil 20 project into a volume enclosed by the body coil 20 and/or into the patient receiving zone 14 of the magnetic resonance device 10. It is conceivable that sections of the connecting elements 34 are led through the body coil 20 and, optionally, also the gradient coil 18, for this purpose. The body coil 20, but also the gradient coil 18, may comprise a recess, which is embodied to house the connecting elements 34.

The connecting elements 34 may comprise one or more electrical connectors and/or cooling connections of the gradient coil 18 and/or the body coil 20. It is also conceivable that electrical connectors and cooling connections are present as separate connecting elements 34 and project into the patient receiving zone 14 at different positions along an inner surface of the patient receiving zone 14.

The connecting elements 34 may be arranged at an end, e.g. an axial end, of the main magnet 12 or the retaining structure 32, to avoid a collision with a patient 15 during a magnetic resonance examination by means of the magnetic resonance device 10.

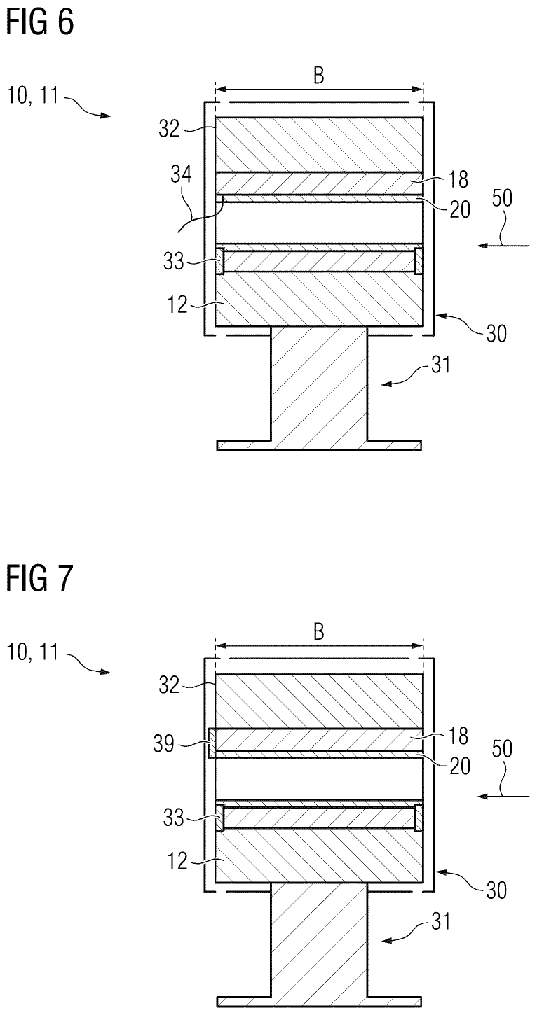

FIG. 6 illustrates an embodiment of the magnetic resonance device 10 according to the disclosure in which a connecting element 34 of the gradient coil 18 is embodied as a flexible connecting element. The flexible connecting element may be embodied to be stowed temporarily within the volume delimited by the retaining structure 32. The flexible connecting element 34 may comprise the electrical connectors required for electrically connecting the gradient coil 18 to the gradient control unit 19 (see FIG. 2). However, the flexible connecting element may also comprise a cooling connection, which is embodied to connect the gradient coil 18 to an external cooling circuit.

In the present example, the flexible connecting element is led through a recess in the body coil 20.

It is conceivable that the body coil 20 also has a flexible connecting element (not shown), which is embodied to connect the body coil 20 electrically to the radiofrequency unit 21. Furthermore, the flexible connecting element of the body coil 20 may also comprise a cooling connection which is embodied to connect the body coil 20 to an external cooling circuit.

FIG. 7 illustrates an embodiment of the magnetic resonance device 10 according to the disclosure having a reversibly removable connecting plate 39. The reversibly removable connecting plate 39 may be embodied to be reversibly disassembled from the retaining structure 32 and/or the field generation unit 11 for transportation purposes to restrict the retaining structure 32 to the width B. The reversibly removable connecting plate 39 may comprise electrical connectors for the gradient coil 18 and/or the body coil 20. However, it is also conceivable that the reversibly removable connecting plate 39 comprises a cooling connection for the gradient coil 18 and/or the body coil 20.

FIG. 8 illustrates an embodiment of the magnetic resonance device 10 according to the disclosure having a rotatable retainer 31. In the present example, the retainer 31 comprises a bearing or an articulated joint, which is embodied to enable the retainer 31 to rotate along the direction of rotation WY. This allows parts of the retainer 31 projecting beyond a width B of the retaining structure 32 to be temporarily rotated or pivoted. For example, the retainer 31 may have a greater dimension along the Z-direction than in the X-direction. By means of the bearing or articulated joint, a part of the retainer 31 having the longer dimension can be temporarily aligned along the X-direction so that a shorter part of the retainer 31 is aligned along the Z-direction and enables the magnetic resonance device to be transported through standardized access paths.

FIG. 9 illustrates an embodiment of the magnetic resonance device 10 according to the disclosure in which the carrier structure 33a of the body coil 20 is housed and fixed in place in an indentation 35 in the wall of the outer vacuum chamber. The indentation 35 may constitute a recess in a material of the wall of the outer vacuum chamber. However, it is also conceivable that the indentation 35 is embodied as a depression or trough which can be obtained e.g. by means of a thermoforming process. At the indentation 35, the wall of the outer vacuum chamber can project in the direction of the main magnet 12 into a volume enclosed by the outer vacuum chamber.

The outer vacuum chamber constitutes a part of the retaining structure 32 of the main magnet 12. In the example shown, the volume delimited by the retaining structure 32 comprises the patient receiving zone 14 enclosed by the retaining structure 32, as well as the vacuum zone enclosed by the retaining structure 32 in which the main magnet 12 and a thermal shield 36 are arranged. In the present case, the main magnet 12 is enclosed on its outer circumference by the thermal shield 36 and an optional cryogenic vessel 37 (in a “wet” magnetic resonance device).

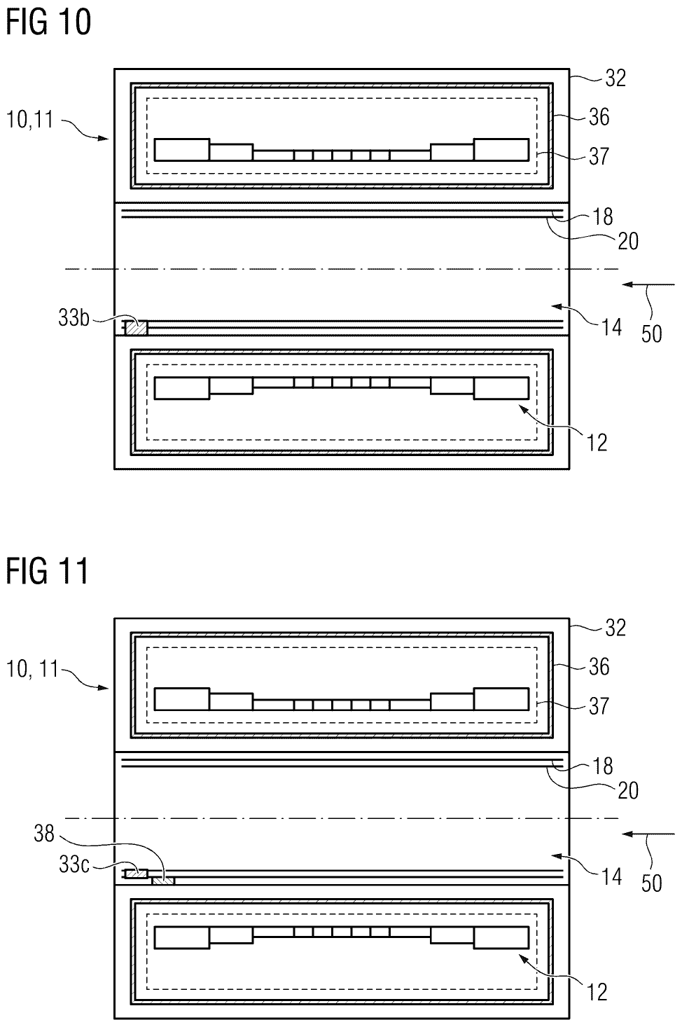

In the present example, the gradient coil 18 has a recess for housing the carrier structure 33a. It is conceivable that the carrier structure 33 only passes through the recess in the gradient coil 18 to secure the body coil 20 to the wall of the outer vacuum chamber. In this case the gradient coil 18 may be mechanically connected separately to the retaining structure 32 or the wall of the outer vacuum chamber by means of a carrier structure 38 (see FIG. 10).

However, the carrier structure 33a may also be embodied to connect the body coil 20 and the gradient coil 18 mechanically to the wall of the outer vacuum chamber.

FIG. 10 shows a further embodiment of the magnetic resonance device 10 according to the disclosure. In the example shown, the carrier structure 33b of the body coil 20 is secured to the wall of the outer vacuum chamber. For this purpose, the carrier structure 33b can pass through a recess in the gradient coil 18 and be mechanically connected to a section of the wall of the vacuum chamber.

The carrier structure 33b may be housed in a recess in the gradient coil 18. The carrier structure 33b may be embodied to secure the body coil 20, but also the gradient coil 18, to the wall of the outer vacuum chamber.

FIG. 11 shows an embodiment of the magnetic resonance device 10 according to the disclosure in which the body coil 20 is mechanically connected to the gradient coil 18 by means of the carrier structure 33c. In this embodiment, the gradient coil 18 may have recesses, which are embodied to house fastening means that connect the carrier structure 33c to the gradient coil 18. The fastening means may for example comprise screws, bolts, pins, rivets, or the like.

In the example shown, the gradient coil 18 comprises a carrier structure 38, which is embodied to connect the gradient coil 18 mechanically to the wall of the outer vacuum chamber. Analogously to an embodiment of the carrier structure 33, the carrier structure 38 can be connected to the wall of the outer vacuum chamber. It is also conceivable that the wall of the outer vacuum chamber has an indentation 35 (see FIG. 9) to accommodate a section of the carrier structure 38 or to enable the carrier structure 38 to be anchored in the indentation 35.

The carrier structure 33c shown in FIG. 11 may also be embodied as a provisional carrier structure, for example. A provisional carrier structure may be embodied to secure the body coil 20 to the gradient coil 18 and/or the retaining structure 32 in a reversible manner. The provisional carrier structure may be embodied to secure the body coil 20 temporarily, e.g. during transportation, to the gradient coil 18 and/or the retaining structure 32. It is conceivable that the provisional carrier structure is embodied to be removed following the transportation and replaced by a conventional carrier structure (see FIG. 1).

Although the disclosure has been illustrated and described in more detail on the basis of the exemplary embodiments, the disclosure is nonetheless not limited by the disclosed examples and other variations may be derived herefrom by the person skilled in the art without leaving the scope of protection of the disclosure.

Independent of the grammatical term usage, individuals with male, female or other gender identities are included within the term.

The various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “circuitry,” “controllers,” “processors,” or “processing circuitry,” or alternatively as noted herein.

What is claimed is:

1. A magnetic resonance device, comprising:
a retaining structure; and
a field generator including a main magnet, a gradient system, and a radiofrequency (RF) system,
wherein the retaining structure is configured to mechanically support the main magnet, and
wherein the field generator comprises an outer circumference that is enclosed by a volume delimited by the retaining structure.

2. The magnetic resonance device as claimed in claim 1, wherein the field generator comprises a connecting element and/or a carrier structure comprising a respective outer circumference that is enclosed by the volume delimited by the retaining structure.

3. The magnetic resonance device as claimed in claim 1, wherein the RF system comprises a carrier structure configured to mechanically couple an RF coil of the RF system to the retaining structure, and
wherein the carrier structure comprises an outer circumference that is enclosed by the volume delimited by the retaining structure.

4. The magnetic resonance device as claimed in claim 3, wherein:
the retaining structure comprises an outer vacuum chamber comprising an outer circumference that encloses the main magnet,
a wall of the outer vacuum chamber has an indentation, and
a section of the carrier structure of the RF system is at least partly housed in the indentation of the outer vacuum chamber.

5. The magnetic resonance device as claimed in claim 1, wherein:
the RF system comprises a provisional carrier structure configured to secure a RF coil of the RF system to (i) a gradient coil of the gradient system and/or (ii) the retaining structure, in a reversible manner,
the provisional carrier structure is configured to be reversibly removable and to secure the RF coil to the gradient coil and/or to the retaining structure, and
the provisional carrier structure comprises an outer circumference that is enclosed by the volume delimited by the retaining structure.

6. The magnetic resonance device as claimed in claim 1, wherein a RF coil of the RF system comprises a connecting element configured to connect the RF coil to (i) a power source, and/or (ii) an external cooling system, and
wherein the connecting element of the RF coil comprises an outer circumference that is enclosed by the volume delimited by the retaining structure.

7. The magnetic resonance device as claimed in claim 1, wherein the field generator comprises a connecting element configured to connect (i) a RF coil of the RF system, and/or (ii) a gradient coil of the gradient system, to (i) an external power source, and/or (ii) an external cooling system, and
wherein the connecting element comprises a flexible connecting element that is configured to be movable relative to the main magnet and to be stowed within the volume delimited by the retaining structure.

8. The magnetic resonance device as claimed in claim 6, wherein the connecting element projects into a volume enclosed by (i) the RF coil, and/or (ii) a patient receiving zone of the magnetic resonance device.

9. The magnetic resonance device as claimed in claim 1, wherein a gradient coil of the gradient system comprises a connecting element configured to connect the gradient coil to (i) a power source, and/or (ii) an external cooling system, and
wherein the connecting element comprises an outer circumference enclosed by the volume delimited by the retaining structure.

10. The magnetic resonance device as claimed in claim 9, wherein a RF coil of (i) the RF system, and/or (ii) the gradient coil, comprises a recess configured to house the connecting element.

11. The magnetic resonance device as claimed in claim 9, wherein the connecting element is led through a recess in a RF coil of the RF system and projects into a volume enclosed by (i) the RF coil, and/or (ii) a patient receiving zone of the magnetic resonance device.

12. The magnetic resonance device as claimed in claim 1, further comprising:
a reversibly removable connecting plate configured to connect (i) a gradient coil of the gradient system, and/or (ii) a RF coil of the RF system, electrically and mechanically, to a power source.

13. The magnetic resonance device as claimed in claim 1, further comprising:
a retainer configured to hold the magnetic resonance device at a predetermined distance from a floor surface,
wherein a part of the retainer that exceeds a dimension of the retaining structure in one spatial direction is reversibly removable.

14. The magnetic resonance device as claimed in claim 1, further comprising:
a retainer configured to hold the magnetic resonance device at a predetermined distance from a floor surface,
wherein the retainer is rotatable and/or pivotable relative to the main magnet.

15. The magnetic resonance device as claimed in claim 1, further comprising:
an outer casing having a reversibly removable section,
wherein user interface circuitry of the reversibly removable section is connected to a control unit of the magnetic resonance device via an electrical interface, and
wherein an electrical connecting lead that connects the user interface circuitry to the electrical interface enables the reversibly removable section containing the user interface circuitry to be reversibly removed from the magnetic resonance device.

16. The magnetic resonance device as claimed in claim 1, further comprising:
an outer casing,
wherein a section of the outer casing encloses the main magnet along a section of a patient access direction, and
wherein a dimension of the section of the outer casing along the patient access direction is less than a dimension of the retaining structure along the patient access direction.
